(19) United States
(12) Patent Application Publication
(10) Pub. No.: US 2007/0161863 A1
(43) Pub. Date: Jul. 12, 2007

(54) Oxygenating laryngoscope

(76) Inventor: Lavern Roxanne Bentt, Takoma Park, MD (US)

Correspondence Address: VORYS SATER SEYMOUR PEASE, 1828 L STREET NW, ELEVENTH FLOOR, WASHINGTON, DC, 20036, US

(21) Appl. No.: 11/646,322

(22) Filed: Dec. 28, 2006

Related U.S. Application Data
(60) Provisional application No. 60/756,578, filed on Jan. 6, 2006.

(51) Int. Cl. A61B 1/267

(52) U.S. Cl. 600/187

(57) ABSTRACT

An improved laryngoscope blade is described wherein the blade includes a conduit for attaching detachable tubing for delivery of oxygen to the airway during laryngoscopy.

### RELATED APPLICATION

This application claims priority from U.S. Provisional Application No. 60/756,578, filed on Jan. 6, 2006.

### FIELD OF INVENTION

The present invention generally relates to the field of medical airway management. More particularly, the present invention relates to a multi-functional oxygenating laryngoscope configured with interchangeable, detachable tubing for oxygen delivery or suctioning of undesirable fluids during intubation or placement of a breathing tube in the windpipe of a patient.

### BACKGROUND OF INVENTION

In medical situations, the need to access the trachea for delivery of oxygen, anesthesia gases, drugs, and for short surgical repair of lesions, arises often. Trained medical personnel utilize a laryngoscope that displaces the tongue and elevates the epiglottis for direct visualization of the glottis and vocal cords, which comprise the entryway to the trachea.

Through the exposed vocal cords one places an endotracheal tube to deliver oxygen, anesthetic gases or resuscitative drugs which are absorbed through the lining of the windpipe or trachea into the circulation system. An inflatable balloon at the lower end of the tube obstructs passage of undesirable material like regurgitated stomach contents around the outside of the tube, and minimizes the risk of stomach contents passing passively into the lungs. Several life saving drugs can be delivered systemically through the mucosa lining of the trachea in code blue situations. In other instances the vocal cords must be exposed by a laryngoscope for quick removal of foreign bodies, lesions or placement of other airway assistive devices.

In the vast majority of cases when the laryngoscope is employed for any of the reasons previously listed, the patient is not breathing. Without active delivery of oxygen, which is mandatory for survival, the user of the laryngoscope is pressured to complete the task in the shortest time possible, often less than three (3) minutes. In a pregnant patient, for example, undergoing general anesthesia for a cesarean section at full term, the combined metabolic rates of mother and fetus lead to rapid consumption of oxygen stores once the once the patient stops breathing from the anesthetic, allowing the anesthetist less than three (3) minutes in most instances to visualize the vocal cords and secure an artificial method of breathing.

In addition, for many patients, abnormalities in anatomy make laryngoscopy difficult. In a comatose person with trauma to the neck, for example, or one who is morbidly obese, the task of identifying the epiglottis, elevating it, and visualizing the vocal cords may require considerably more time than is safe in a non-breathing patient rapidly consuming oxygen stores. The possibility of brain injury from lack of oxygen then becomes very real. Several techniques have evolved in an attempt to alleviate these concerns.

For example, one technique involves administering a hollow shaft laryngoscope attached to a hollow handle, which allows the placement of tubing to suction smoke and steam out of the airway during specifically laser procedures for removal of airway lesions, masses and growths. Another technique involves administering a laryngoscope blade having conduits permanently attached to or incorporated into the body of the blade.

More specifically, Durrant, U.S. Pat. No. 2,854,004, discloses a straight laryngoscope blade with a suction head mounted on the tip or distal end of the blade together with conduit means extending from the head to a point adjacent the proximal end of the blade where a suitable connection to a vacuum pump or the like may be made. The suction head is non-detachable and is in the form of a “T” shaped metallic tubing molded permanently into the body of the blade. The object of Durrant's invention is to provide suction for the removal of mucus or to provide reduced air pressure at the tip of the blade. However, as the suction head is mounted on the distal end of the blade, it projects slightly beyond the tip creating an increased risk of trauma to the fragile glottic tissues. Further, although this device leaves one hand of the user advantageously free, the device is bulky and has an unwieldy tip that can only be used for suctioning. Moreover, this device is difficult to manufacture because the placement of the metallic tubing requires the creation of a slot at the proximal end of the blade through which the tubing can enter before continuing on down the length of the blade. Also, the distal tip of the blade needs to be cut away to allow for placement of the bulky distal suction head.

May, U.S. Pat. No. 4,126,127, discloses a laryngoscope blade that comprises an illuminated, molded tongue retractor, wherein, as viewed by a patient, a suction conduit extends up the left side of the blade, from just short of the distal tip of the blade, to the proximal end of the blade, with means for attachment to a separate suction hose and vacuum pump. A flow of 100% oxygen is provided near the distal tip of the blade through a second conduit, smaller in diameter than the first, extending down the right side of the blade from the proximal end of the blade, with means for attachment to a separate oxygen delivery hose and oxygen source. The improved blade is uniquely shaped to facilitate endotracheal intubation, in a 100% oxygen environment, with high-volume suction immediately available, at the glottis, in the illuminated field of view. However, this blade presents several difficulties in use and manufacture. The apparatus has two permanent conduits. The two conduits create a bulky device which further obstructs the view when the laryngoscope blade is inserted into the oropharynx. Bulky devices are also more difficult to place accurately and without trauma to the surrounding tissues. The permanent conduits can not be removed for cleaning, making sterilization difficult, costly and time consuming. The blade is further burdened by two sets of hose connections, which leads to diminished mobility of the blade during use, less ease of use during an emergency, and a higher likelihood of erroneously disconnecting the wrong hose.

Cook, Jr., U.S. Pat. No. 6,248,061, discloses a suctioning laryngoscope blade that allows a user to suction the airway of a patient during use. More particularly, Cook, Jr. discloses a suctioning laryngoscope blade that includes a blade portion and a suction tube, coupled to an external surface of the blade portion, wherein the suction tube is a permanent component of the laryngoscope blade. According to Cook, Jr., the suction tubing is externally fixed and permanently incorporated into the body of the blade in a non-detachable fashion. There is a suction end at the distal tip of the blade and an adaptation end at the proximate tip that is to be attached to a vacuum source. The device is specifically designed to improve suctioning of secretions during visualization of the oropharynx and intubation of the trachea. However, the metallic tubing employed is non-detachable making sterilization of tubing that is used primarily for evacuation of undesirable bodily fluids a significant issue. Further, the tubing is placed visibly on the upper surface of the blade, which has the potential for obstructing the user's view. Moreover, the tubing is permanently incorporated and can not be removed or retracted to improve the view. Lastly, the tubing of Cook, Jr., is incorporated into the curved blade of the laryngoscope.

Urbanowicz et al., U.S. Pat. No. 5,897,489, discloses a suction tube for use with a laryngoscope comprising a suction tube configured in the general profile of a laryngoscope, having an elongated clip which may be snapped on to the handle of a laryngoscope and easily removed after use. The device enables the user to work with the laryngoscope, the suction tube, and an airway tube all at the same time to accomplish oral intubation efficiently and without distractions. Urbanowicz et al. essentially discloses long, flexible, disposable tubing specifically for the application of vacuum suctioning of fluids from the glottis during intubation of the trachea. It may be temporarily fixed to the handle of the laryngoscope via an elongated clip that runs most of the length of the handle. The purpose of Urbanowicz et al. is to provide a means for quick evacuation of material from the airway without having to change hands or be encumbered with a suction hose in the hand that is meant to manipulate the necessary endotracheal tube. There is a cord device running most of the length of the tubing via an inner channel whose manipulation via a distal attached ring moves the distal tip of the tubing into multiple positions. A large lever projects out from the handle and moves the cord through the tubing. However, this design is bulky, preferably disposable, and intended to suction only. This design also has the potential for equipment failure because of the slender cord device and the dependence on manipulation of the tip.

Bartlett, U.S. Pat. No. 4,947,896, discloses a laryngoscope that has a blade and a removably attached handle, which can be fixed to the blade at a desired angle. The blade has a predetermined cross-section of compound curvature defining a plurality of channels, including a viewing channel and a plurality of service channels. The blade also supports twin halogen lights which project light down opposing axial sides of the blade structure. Electric wires run in the service channels between the lights and batteries received in the handle. An adjustable position suction tube is entrained in the blade in another service channel, with an adjustment actuator supported on the handle. A roughened surface is formed in a tongue contact area of the blade to enhance frictional engagement of the patient's tongue. However, this invention changes the basic nature of the laryngoscope blade and is complicated to make and use with its multiple channels and electrical wires.

An overall drawback of these devices is that they are complicated and bulky, which makes manipulation difficult. In addition, the existing devices are not detachable for easy sterilization. Furthermore, many of these devices do not provide for the efficient delivery of oxygen.

There is therefore a need for a laryngoscope device that is easily assembled and disassembled with facility and speed. There is also a need for such a device whose components are detachable for sterilization and storage. There is also a need for such a device that can efficiently deliver oxygen to the trachea.

### SUMMARY OF INVENTION

The present invention satisfies, to a great extent, the foregoing and other needs not currently satisfied by existing laryngoscope blades. This result is achieved, in a preferred embodiment, by an improved oxygenating laryngoscope blade that allows for the concurrent delivery of oxygen to the patient's trachea while the blade is in use to elevate the glottis and to deliver a view of the vocal cords for placement of the endotracheal tube or other critical task.

The present invention relates to a novel modification of a standard straight laryngoscope blade commonly used in the area of medical airway management that allows the active delivery of supplemental oxygen during the process of looking into the mouth and oropharynx for identification of the vocal cords and entryway into the windpipe.

Laryngoscopy can be complicated by lack of or potential lack of oxygen before the process is complete, the airway is secured and continuous delivery of oxygen to a non-breathing person is established. The present invention involves the application of secure, non-detachable metallic rings that create a conduit for tubing that can deliver oxygen. According to a preferred embodiment, the tubing is preferably metallic, more preferably metallic tubing with a specific diameter with a proximal connection to oxygen, wherein the diameter of this connection is preferably variable to allow for the attachment of most medical grade oxygen carrying tubing.

Accordingly, it is an object of the present invention to make the process of placing an endotracheal tube or conducting other tasks in the airway safer.

It is another object of the present invention to provide a laryngoscope that is used primarily for oxygen delivery.

It is another object of the present invention to provide a preferably continuous, directed flow of oxygen into the open airway during laryngoscopy with a straight blade.

It is another object of the present invention to extend the amount of time a patient is permitted to be non-breathing, by delivering oxygen directly to the trachea during laryngoscopy.

It is another object of the present invention to provide a laryngoscope that provides effective oxygenation of the glottis by providing a flow or stream of oxygen that is delivered directly to the opening in the airway to the trachea where it is useful.

It is another object of the present invention to provide a new and improved laryngoscope blade that is easy to manufacture and therefore cost efficient to make.

It is another object of the present invention to provide an improved laryngoscope blade with no significant alteration to the shape or contour of the blade and minimal change in the weight.

It is another object of the present invention to provide a laryngoscope blade that is simple to manufacture by having no need for additional modifications to the blade by cutting away a slot or changing the basic contour of the blade at either the proximal end or the distal end.

It is another object of the present invention to provide a simple and functional construction design wherein tubing, preferably oxygenating tubing, remains hidden under the curved flange of the straight laryngoscope blade to avoid any obstruction in the line of sight along the blade.

It is another object of the present invention to provide a laryngoscope blade that comprises at least two clips that form a conduit for, and secure tubing of choice to the upper surface of the blade.

It is another object of the present invention to provide a laryngoscope blade comprising at least two clips hidden on the blade that allow for easy manipulation and detachment of the tubing for sterilization.

It is another object of the present invention to provide a laryngoscope blade that comprises at least two non-detachable clips that are hidden under the curved flange of the blade for the attachment of tubing such that when tubing is inserted into the clips, the tubing conforms to the natural shape of the blade thereby avoiding any possibility of obstructing the user's view.

It is another object of the present invention to provide a laryngoscope blade wherein tubing is positioned under the curved flange of the blade such as to allow tubing of a larger diameter which affords more effective oxygen flow rates without sacrificing visibility.

It is another object of the present invention to provide a laryngoscope blade with tubing, preferably oxygenating tubing, wherein the distal tip of the tubing follows the slightly curved line of the blade and does not project beyond, thereby minimizing the risk of trauma to the surrounding tissues from said tubing.

It is another object of the present invention to provide a detachable or non-detachable graduated connector tip on the proximal end of the laryngoscope blade that preferably extends a few millimeters beyond the end of the blade for attachment of oxygen supply tubing or vacuum suction.

It is another object of the present invention to provide a laryngoscope blade comprising tubing that can be used for suctioning or for delivery of oxygen, preferably for delivery of oxygen into the windpipe during periods when the patient is least capable of doing so spontaneously.

It is another object of the invention to provide for a laryngoscope with preferably detachable tubing that allows for easy sterilization of the blade and of the optionally non-disposable tubing.

It is another object of the invention to provide a laryngoscope with tubing that may be retracted to improve visibility while still being close enough to the glottis to deliver oxygen directly into the trachea.

The above and other features and advantages are achieved through the use of a novel laryngoscope as disclosed herein. There has thus been outlined, rather broadly, the more important features of the invention in order that the detailed description thereof that follows may be better understood, and in order that the present contribution to the art may be better appreciated. There are, of course, additional features of the invention that will be described further hereinafter.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that equivalent constructions insofar as they do not depart from the spirit and scope of the present invention, are included in the present invention.

For a better understanding of the invention, its operating advantages and the specific objects attained by its uses, reference should be had to the accompanying drawings and descriptive matter which illustrate preferred embodiments of the invention.

### BRIEF DESCRIPTION OF THE DRAWINGS

FIG. 1 is a perspective view of a laryngoscope blade according to one embodiment of the present invention.

FIG. 2 is a side view of the laryngoscope blade shown in FIG. 1.

FIG. 3 is a side view of the laryngoscope blade shown in FIG. 1

FIG. 4 is a cross-sectional view taken about lines 4-4 of FIG. 3.

FIG. 5 is a side view of the laryngoscope blade shown in FIG. 1 in a patient's trachea.

### DETAILED DESCRIPTION OF PREFERRED EMBODIMENTS

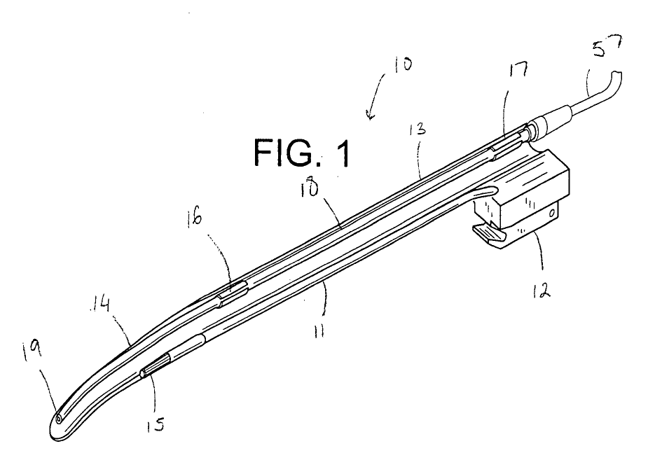

Referring now to the figures, wherein like reference numerals indicate like elements, in FIG. 1 there is shown a side view of the laryngoscope 10.

The laryngoscope 10 is comprised of two connected and easy to disassemble parts: a battery-containing handle (not shown), and a light-emitting blade 11. The handle is preferably a cylindrical structure that attaches to the blade 11 at one end via a “hook on” device 12 in a manner well-known described in the prior art, see for example Palmeter, U.S. Pat. No. 2,433,705 or Bar-Or, U.S. Pat. No. 5,702,351. The bottom end of the handle has a detachable cap which allows access to a cavity inside the handle for battery placement. Alternatively, the illumination could be transmitted by a fiber optic bundle, also known in the prior art but not depicted in the drawings and not considered crucial to the present invention. However, this alternative method of illumination is mentioned to illustrate that a variation of the method of illumination of the suctioning/oxygenating laryngoscope blade of the present invention is not to be considered a departure from the spirit of the present invention. The method of illumination of the blade, and its exact spatial arrangement, is subject to change with the development and improvement of the present invention.

The blade 11 has a proximal end 13 and distal end 14. At the distal end 14 of the blade is a light source 15 provided by a bulb that is used to illuminate the field of view under consideration. The handle powers the light source 15 at the distal end 14 of the blade 11. The proximal end 13 of the blade can attach to and detach from the handle, which houses the battery by means of a “hook on” 12 or other suitable connection well-known to one of ordinary skill in the art. The “hook on” connection is standard and offers the possibility of attaching a variety of shapes and sizes of blades and handles.

The laryngoscope 10 comprises at least two non-detachable clips, one at the distal end 16 and one at the proximal end 17, which form a conduit for and secure the removable tubing 18 of choice to the upper surface of the blade.

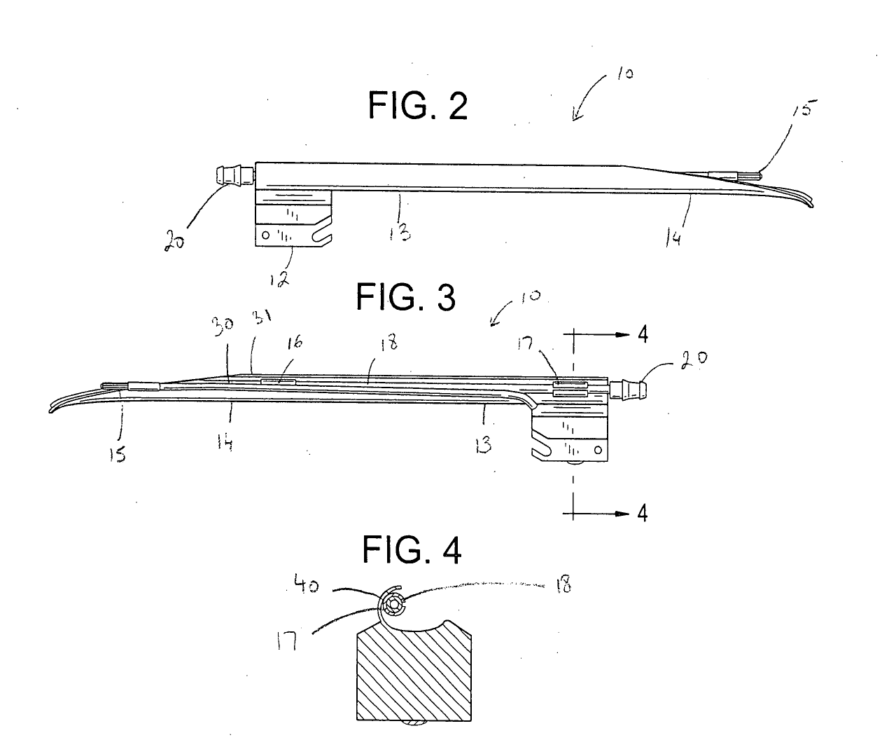

As shown in FIG. 2, a graduated connector tip 20, which may be detachable or non-detachable, is located on the proximal end 13 of the laryngoscope blade and preferably extends a few millimeters beyond the end of the blade for attachment of oxygen supply tubing or vacuum suction 57.

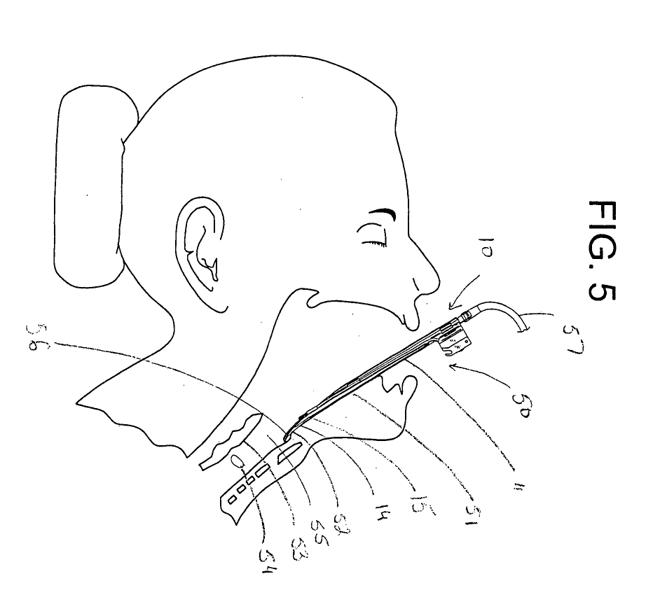

As seen in FIG. 4, the clip 17 of the laryngoscope blade is hidden under the curved flange 40 of the blade for the attachment of tubing 18 such that when tubing is inserted into clips 16 and 17, the tubing conforms to the natural shape of the blade thereby avoiding any possibility of obstructing the user's view.

To use the laryngoscope 10, first, the blade 11 is attached to the handle and the device is placed in a functional position where the battery connection is made. The light 15 on the distal tip 14 of the blade 11 is powered. The handle is held firmly in the user's left hand with all fingers wrapped around the cylindrical body of the handle, and the blade 11 held below it.

As shown in FIG. 5, the blade 11 is placed into the mouth 50, on top of the tongue 51, and displaced from the line of view and advanced in until the distal, lighted 14, 15 tip of the blade 11 sits securely on the undersurface of the epiglottis 52.

The distal tip 14 of the non-flexible blade is used to elevate the epiglottis 52 off the glottis 53, thus exposing the vocal cords 54 and the entryway into the trachea 55 to the user's view. The user than places an endotracheal tube into the trachea 55 through the open vocal cords 54, administers an appropriate drug, places a device or removes a device or lesion.

During this process, in a planned anesthetic, the patient is immobile and not breathing. The anesthetic drugs administered eliminate the ability to breathe and in most cases also intentionally paralyze the muscles. Oxygen is provided to the patient before administration of the medications which create unconsciousness are given. Usually, delivering oxygen before the administration of the anesthetic drugs creates an adequate supply of critical oxygen stores subsequently used to fuel the brain and vital organs while the patient is unable to breathe. In some not uncommon instances it is difficult for the user to visualize the desired structures requiring time for manipulation of the blade into appropriate position. In some instances the oxygen provided before the patient stops breathing at the onset of laryngoscopy in not adequate for the patient's needs because of a high metabolic rare or poor oxygen storage capacity supply. During these times the level of oxygen in the body falls precipitously, and may become critically and life threateningly low.

The laryngoscope blade of the present invention allows for the concurrent delivery of oxygen while the blade is in use to elevate the epiglottis and deliver a view of the vocal cords. Instead of the customary three minutes, during which time the average patient is permitted to be apneic before available oxygen falls to dangerously low levels, the laryngoscope of the present invention allows for a longer period of time of apnea because of the simultaneous delivery of oxygen. The present invention makes the process of placing an endotracheal tube or working in the airway a much safer one.

The laryngoscope of the present invention may also be used to deliver local anesthetic to the airway, emergency drugs like lidocaine and atropine, and also to supply oxygen while a short repair of an airway problem is underway.

The blade of the present invention may be constructed straight or with some amount of curve. According to a preferred embodiment, the laryngoscope blade of the present invention is preferably a predominantly straight blade, wherein the leading one inch of the distal tip 14 of the blade 11 is preferably upturned slightly 56 (as viewed by a patient). The blade may be constructed in different sizes for different size patients, such as, for example, for infants, children and adults.

The laryngoscope blade may be constructed of metal or some other material such as plastic, or a combination of materials that provides sufficient structural strength. According to a preferred embodiment, the laryngoscope blade is made of stainless steel.

During laryngoscopy and endotracheal intubation, if the distal portion 30 of the tubing impairs visibility, the tubing may be retracted towards the proximal end 13 until the tip of the tubing 19 is under the curved distal end of the flange of the blade 31, improving visibility while still being close enough to the glottis to deliver 100% oxygen directly into the trachea.

The clips 16 and 17 allow the attachment of any tubing. The graduated connector tip 20 may be attached to oxygen supply tubing or vacuum suction 57. Standard issue oxygen supply tubing, jet ventilator tubing or custom suction tubing may be used and are well known in the art. The chosen tubing can easily be connected to one light weight standard issue hosing attached to an oxygen source or a vacuum suction source. According to a preferred embodiment, the tubing that delivers oxygen has a moderately large diameter to deliver a greater volume of oxygen while still remaining hidden under the flange 31 and 40 of the laryngoscope.

In a preferred embodiment, the tubing may be metallic tubing, standard issue PVC oxygen delivery tubing available in any operating room, tubing attached to a jet ventilator, a device that delivers oxygen to the airway in pulses at high pressure, or other suitable tubing well known in the art.

It can be appreciated that variations to the present invention would be readily apparent to those skilled in the art, and the present invention is intended to include those alternatives. Accordingly, the scope of the present invention should be assessed as that of the appended claims and any equivalents thereto.

What is claimed is:

1. A laryngoscope blade comprising a conduit for attachment of tubing.

2. The blade according to claim 1, wherein the tubing delivers oxygen during laryngoscopy.

3. The blade according to claim 1, wherein the tubing is detachable.

4. The blade according to claim 1, wherein the conduit comprises at least two clips.

5. The blade according to claim 4, wherein the clips are non-detachable.

6. The blade according to claim 4, wherein the blade has a curved upper flange and the clips are hidden under the curved flange of the blade.

7. The blade according to claim 1, wherein the tubing is retractable.

8. The blade according to claim 1, wherein the blade is made of a material selected from the group consisting of metal or plastic.

9. The blade according to claim 1, wherein the tubing is made of a material selected from the group consisting of metal or plastic.

10. The blade according to claim 1, wherein the tubing is of equivalent length and curvature of the blade.

11. The blade according to claim 1, wherein the blade has a proximal end and a distal end and the proximal end is attached to one of an oxygen source, a jet ventilation source or a suction source.

12. The blade according to claim 11, wherein the source is attached to the proximal end via a graduated connector tip.

13. The blade according to claim 1, wherein the conduit can accommodate tubing of various diameters.

14. The blade according to claim 1, wherein the blade is infant size, child size or adult size.

15. The blade according to claim 1, wherein the blade is a straight blade.
